(19) United States
(12) Patent Application Publication
(10) Pub. No.: US 2023/0073081 A1
(43) Pub. Date: Mar. 9, 2023

(54) SWIVEL DEVICE FOR AN ULTRASOUND PROBE

(71) Applicant: SUPERSONIC IMAGINE, Aix-en-Provence (FR)

(72) Inventors: Morgane LE GARREC, Aix-en-Provence (FR); Damien MAJOREL, Ales (FR); Jean-Alain MARTIN, Pertuis (FR)

(73) Assignee: SUPERSONIC IMAGINE, Aix-en-Provence (FR)

(21) Appl. No.: 17/941,546

(22) Filed: Sep. 9, 2022

(30) Foreign Application Priority Data
Sep. 9, 2021 (FR) 2109429

(51) Int. Cl. A61B 8/00; A61B 5/00

(52) U.S. Cl. CPC A61B 8/4444; A61B 5/6843

(57) ABSTRACT

This disclosure concerns a device for an ultrasound probe for acquiring ultrasound data from a medium, wherein the device is configured to be linked to the probe in a swiveling manner, so that the angle between the probe and the surface of the medium can be adjusted and/or set by swiveling the device.

### TECHNICAL FIELD

This invention concerns medical systems and the procedures using such systems. A system of this kind can constitute a medical examination system. More particularly, it can constitute an ultrasound system.

### PRIOR ART

An ultrasound system usually includes electronic means, for example, a sensor and/or an ultrasound probe for acquiring the patient's data, and a processor for processing the data acquired.

An ultrasound system can be used for supplying data from a medium for examination. Examples of such systems include an optical imaging system, an ultrasound imaging system, an x-ray system, a computer tomography system, a mammography system, among others. With regard to medical applications, the medium is a body, for instance, a part of the patient's body (muscles, fetus, chest, liver, abdomen, etc.).

In general, the probe is held against the surface of an examined medium so as to acquire data on this medium. In this context, it is important not to shift the probe during the acquisition of data or only shift it in a specific and desired manner.

For example, in the case of a classic ultrasound imaging in mode B (“Brightness mode”), it is desirable that the probe remains in a fixed position during the acquisition of data.

In addition, there are ultrasound imaging methods in which a deformation is applied to the medium during the acquisition of data as, for instance, described by WO2021116326A2. In this method, the non-linear elasticity of a medium is quantified by using shear waves—“non-linear shear wave elastography” (NL-SWE). The method includes the following steps: A1. Collecting a succession in time of shear wave elasticity data from the medium; A2. Application of a successively changing deformation to the medium according to a predetermined sequence of deformations during the collection of shear waves; A3. Observation of the evolution of real deformation; and B. Quantification of the non-linear elasticity of the medium as a function of the temporal succession of data and the evolution of deformation.

Therefore, in the NL-SWE mode, it can be desirable that the probe remains in a fixed position during the acquisition of data.

It can be difficult, however, to avoid all undesirable movement of the probe during the acquisition of data, especially during the deformation step. For example, the ultrasound gel used can make the medium surface slippery. Likewise, a hard lesion in the medium or the respiration of the patient can lead to undesirable shifts.

Therefore, undesirable shifts can notably lead to a transversal shift of the probe (e.g., sliding on the surface of the medium) and/or a rotation shift modifying the tilt angle.

In addition, given that force is applied solely to the surface of the probe during deformation, it is difficult to achieve such deformation's homogeneity.

Moreover, the initiation of data acquisition often requires an action by the user on the user interface (e.g., by pressing a button) synchronized with the beginning of the deformation.

There are various accessories for ultrasound probes available. For instance, EP3202326A1 describes an ultrasound probe accessory. The accessory includes an element of acoustic adaptation envisaged on a transmitter-receptor of an ultrasound probe and deformed on the basis of a surface of an inspection target when the ultrasound probe sweeps the surface of the inspection target.

The document EP1629777A1 also discloses a pressure mechanism for applying pressure to the contact surface of a subject perpendicularly to the surface of emission/reception of ultrasound waves of an ultrasound probe via the surface of emission/reception of ultrasound waves.

However, the available accessories are not adapted for addressing the aforesaid issues, in particular, for applying homogeneous pressure on the medium's surface and simultaneously maintaining the probe at a selected angle in relation to the medium's surface.

Invention Disclosure

The purpose of this disclosure is, therefore, to provide a device for an ultrasound probe that allows holding the probe at an angle selected by the user during the acquisition of data, and ensures a homogeneous deformation of the medium during the acquisition of data by maintaining the selected angle.

This disclosure concerns an (accessory) device for an ultrasound probe for acquiring ultrasound data from a medium, wherein the device is configured to be linked to the probe in a swiveling manner, so that the angle between the probe and the medium's surface can be adjusted and/or set by swiveling the device.

Thanks to this device, the user can stabilize and maintain the probe at a predefined angle/tilt during the acquisition of data.

For instance, with regard to a classic ultrasound imaging in mode B (“Brightness mode”), it is desirable that the probe remains in a fixed position during the acquisition of data. According to the location of the area of interest of the medium which should be imaged, it can also be helpful if the probe is not placed perpendicularly to the medium's surface but with a selected tilt angle. In this case, the device of this disclosure ensures that the tilt angle is maintained during the acquisition of data.

According to another example, in the NL-SWE mode, it can be desirable to place the probe with a tilt angle selected in relation to the medium in order to acquire data from a specific area interest, such as a lesion. Subsequently, the device of this disclosure allows to retain the selected tilt angle during the deformation step(s).

In particular, the device can be configured to be linked to the probe in a swiveling manner, so that when (or while) the device is placed against (and/or maintained on) the surface of the medium observed, the angle between the probe and the surface of the examined medium can be adjusted and/or set by swiveling the device.

The ultrasound probe can cooperate with a system which is configured to process data from a medium acquired by the probe.

The device can be configured to be positioned and/or attached to the probe in a swiveling manner. Thus, “linking to the probe in a swiveling manner” can mean according to an example “positioned and/or attached to the probe in a swiveling manner.”

The device can also include adaptable means configured for attaching the device to the bodies of different probes.

The probe can be configured to emit ultrasound waves and/or can include a transducer configured to emit ultrasound waves on a primary side of the probe.

The device can be configured to be positioned and/or attached on the primary side of the probe in a swiveling manner. The device can, therefore, be configured to be positioned and/or attached, for instance, on the body of the probe and enabling the swiveling of the latter.

Alternatively, the device can be configured to be positioned and/or attached on another side than the primary side of the probe in a swiveling manner. This configuration can be applied, for example, if the probe is configured for intracavitary areas. For instance, the device can be configured to be positioned and/or attached to the handle of the probe or adjacent to the handle. Alternatively, the device can be configured to be positioned and/or attached in a swiveling manner on a side opposite to the primary side of the probe.

The angle can be understood as a tilt angle between the direction of the ultrasound waves emitted by the probe and the medium's surface.

The device can comprise an opening configured for enabling the transmission of ultrasound waves. Thus, the opening can be configured so that the emission and reception of ultrasound waves would be possible without disruption.

The device can comprise a plate and/or a flat surface configured to be placed against the medium's surface and to stabilize the angle between the probe and medium's surface.

In modes similar to the NL-SWE mode, for instance, the device can, therefore, ensure a homogeneous deformation of the medium and consequently provide a good image quality. In fact, during the deformation with a probe without the device, only the tissue area situated directly under the probe will be compressed, which can disrupt the estimation of the deformation if the imaging plane varies during the acquisition.

The device and/or its opening can include a means of coupling configured for enabling the transmission of ultrasound waves.

The means of coupling can include a film and/or gel pad, and optionally an adhesive for attaching the film and/or gel pad to the device.

According to one example, the means of coupling can include a bi-material film with one or several adhering parts positioned in a peripheral area. The bi-material film can also include a part configured to couple the device to the medium and/or enable the transmission of ultrasound waves. This latter part can be positioned in a section where the ultrasound waves are emitted by the probe.

The device and/or its opening can include a means of stabilization configured for optimizing the stability of the device on the medium's surface.

The means of stabilization can include an adhering part on a surface of the device configured to be placed against the surface of the medium.

The means of stabilization can, therefore, prevent the probe and the device from sliding on the surface of the medium. The device can thus ensure an acquisition of data without undesirable movement of the probe in relation to the medium during the acquisition, such as a rotation (prevented by the optional plate of the device) and a transversal shift (prevented by the optional means of stabilization of the device).

The device can be configured to adjust and/or set an angle by using mechanical and/or electromechanical, and/or electronic means.

Mechanical means can include a clutch for adjusting and/or setting the angle.

The device can be configured for adjusting the angle in steps or continuously, for example, by controlling the clutch.

The device can also comprise a temperature sensor configured for measuring the temperature of the medium's surface and/or on the probe's surface.

The device can also comprise a pressure sensor configured for measuring the pressure that the device applies to the medium's surface.

The device can also comprise a pressure indicator configured to indicate a level of pressure applied to the medium's surface.

The device can also comprise multiple local pressure sensors. Each local pressure sensor can be configured to measure the pressure applied to the medium's surface by a different section of the device. The device can also optionally include multiple local pressure indicators, with each of them being configured to indicate a local pressure level.

The pressure indicator and/or local pressure indicators, and/or one or several temperature indicators can be placed on an opposite side of a free surface of the device, which is placed against the medium.

The device can also include a tilt sensor configured to measure the angle between the probe and the medium's surface.

The device can also include a display for indicating such angle.

The device can also include a position guiding system configured to indicate a target position to the user of the probe towards the target position.

The position guiding system can comprise direction indicators located on the free surface of the device and configured to indicate a direction towards the target position.

The position guiding system can also comprise means of localization configured to localize the current position of the device in relation to the medium.

The position guiding system can be configured to communicate with an external processing unit to transmit localization data and/or receive data of a target position and/or of a direction towards the target position. This external processing unit can constitute, for example, a system that collaborates/communicates with the ultrasound probe and that is configured to process data from a medium acquired by the probe.

The device can also include an ergonomic support, such as a pad, configured so that a hand holding the probe can rest on it.

This disclosure also concerns an ultrasound probe for acquiring ultrasound data from a medium. The probe can be configured to cooperate and/or communicate with a system intended for processing ultrasound data. The probe can include a device according to this disclosure.

This disclosure also concerns an ultrasound system, including a device according to this disclosure. The system can optionally comprise an ultrasound probe to acquire data from a medium. The position guiding system can also include means of localization configured to localize the position of the device in relation to the medium.

Such system can constitute, for example, a system that cooperates and/or communicates with the ultrasound probe and that is configured to process data from a medium acquired by the probe.

This disclosure also concerns a process for acquiring data through an imaging probe by using a device linked to the probe in a swiveling manner, including the following steps:

A. adjusting and/or setting the angle between the probe and the medium's surface by swiveling the device;

B. acquiring data from the medium by using the probe.

Subsequently, the angle can first be adjusted and/or set. Then, data can be acquired by the probe. Thus, it is possible to acquire data at a stable tilt angle.

Steps A and B can be repeated.

The process may further include the following steps:

C1. changing a level of deformation of the medium applied by the device, and/or

C2. changing the angle.

Steps C1 and/or C2 can be realized after step B.

Step B can be repeated after step C1. Subsequently, by changing the level of deformation according to step C1, as well as by repeating step B, data can be acquired at two different deformation levels.

In the event where steps B and C1 are repeated, the data can be acquired at more than two different deformation levels. Such a method can be useful, for example, in the NL-SWE mode.

Step B can also be repeated after step C2. Subsequently, by changing the tilt angle according to step C2, as well as by repeating step B, data can be acquired at two different tilt angles of the probe.

In the event where steps B and C2 are repeated, data can be acquired at more than two different tilt angles. Such a method can be useful, for example, in a 3D image collection mode, which is based on the acquisition of a 2D-image data set of a medium at different angles of the probe in relation to the medium.

Optionally, the process can also include a reiteration of steps A, B, C1, and/or C2, wherein, in the supplementary step A, the angle is adjusted and/or set before the data is acquired in step B.

However, an adjustment and/or setting of the angle can also be already carried out in step C2. Therefore, step C2 can also be understood as a reiteration of step A.

The characteristics and advantages of the invention will appear upon reading the description which follows, given solely by way of non-exhaustive example, and made with reference to the accompanying Figures. In particular, the examples illustrated in the Figures can be combined, unless there is any significant inconsistency.

### BRIEF DESCRIPTION OF FIGURES

FIG. 1 schematically shows a conventional probe in a first lateral view.

FIG. 2a schematically shows a perspective view of the first embodiment of the device according to this disclosure.

FIG. 2b schematically shows a first sectional view of the device in FIG. 2a, wherein a movement of swiveling of the device is illustrated.

FIG. 3a schematically shows a first lateral view of the second embodiment of the device according to this disclosure, wherein the device can have a coupling film.

FIG. 3b schematically shows a first lateral view of the second embodiment of the device according to this disclosure, wherein the device can have a gel pad.

FIG. 4a schematically shows a second lateral view of the fourth embodiment of the device according to this disclosure, wherein the device can have a means of stabilization configured to optimize the stability of the device on the medium's surface.

FIG. 4b schematically represents a view from below of the device in FIG. 4a.

FIG. 5 schematically shows a first lateral view of the fifth embodiment of the device according to this disclosure, wherein the device can have a mechanical means for a swiveling by steps/levels and/or by indexing.

FIG. 6a schematically shows a second lateral view of the sixth embodiment of the device according to this invention, wherein the device can be configured for a continuous and lockable swiveling in any position.

FIG. 6b schematically shows a first lateral view of the device in FIG. 6a with an optional tilt sensor.

FIG. 6c schematically shows a view from above of the device in FIGS. 6a and 6b with an optional tilt sensor.

FIG. 7a schematically shows a first lateral view of the seventh embodiment of the device according to this disclosure, wherein the device can have pressure sensors and pressure indicators.

FIG. 7b schematically shows a view from above of the device in FIG. 7a and in particular the positioning of the pressure indicators.

FIG. 8a schematically shows a first lateral view of the eighth embodiment of the device according to this disclosure, wherein the device can have a positioning sensor and direction indicators.

FIG. 8b schematically shows a view from above of the device in FIG. 8a.

FIG. 9 schematically shows a first lateral view of the ninth embodiment of the device according to this disclosure, wherein the device can have an ergonomic support allowing the resting of the wrist.

### DESCRIPTION OF THE EMBODIMENTS

Across the various Figures provided for illustrative purposes, the same numerical references denote the same or similar elements. The different embodiments can be combined in any way, unless otherwise specified.

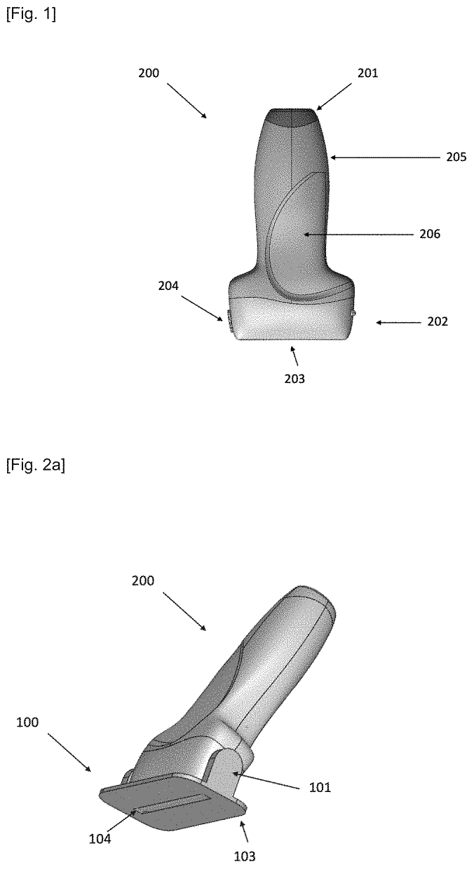

FIG. 1 schematically illustrates a conventional probe in a first lateral view. Such probe 200 can be used (that is equipped or linked) with a device according to this disclosure (not illustrated in FIG. 1).

The probe is configured to observe a medium by acquiring data from the medium, for example, from a human body or animal. For instance, the probe can be an ultrasound probe. In other words, the probe can include one or several ultrasound transducers. However, the probe can also include another type of imaging sensor. The probe can then also constitute an imaging probe in any application, which requires a tilt and/or stable position of the probe in relation to the medium observed during the acquisition of data. According to another example the probe can include one or several laser optic devices. Such a probe can also be configured for an optoacoustic imaging using ultrasound transducers and laser optic devices.

Generally, the probe can be configured for a diagnostic and/or therapeutic use. For example, the probe can be configured to acquire data from a medium, such as for imaging of the medium, in order to provide for a diagnosis. Moreover, the probe can be configured for a non-invasive process of focusing acoustic waves in a medium, for instance, a dissipative heterogeneous medium, including a substantially homogeneous medium (e.g., the brain) surrounded at least partially by a dissipative aberrating layer (e.g., the skull). Acoustic waves can be emitted from the exterior of the aberrating layer and focused in the substantially homogeneous medium.

Furthermore, the probe can be configured to enable the visualization and guiding of the insertion of a biopsy needle in real time. For example, the probe can cooperate with a needle guide which can, for example, attach itself to the probe for the guiding of the needle. The data acquired by the probe can, in particular, be used to monitor the biopsy. According to another example, the navigation of the needle can superimpose in real time ultrasound and other imaging modes on volumes originating from sectional modalities (IRM, CT, PET, or 3D-US).

The probe can also be used to monitor and/or guide the implementation of biopsy markers in the medium.

The probe can have different forms and/or networks of transducers. For instance, the probe can be a bi-dimensional probe (e.g., with a linear network of transducers), a “1.5D” probe (e.g., including several types of transducers thus offering different functions, such as a first type of transducers in the form of a linear network allowing an ultrasound image, and a second type of transducers intended to generate an internal mechanical constraint allowing propagation of a shear wave in the medium, and placed linearly either side of the first type of transducers), an “asymmetrical” probe (e.g., with a network with a number of elements much more important in one dimension than in the other), a tri-dimensional probe (e.g., with a network with the same number of elements in the two dimensions), a curved probe, a matrix probe, or an intracavitary probe. The probe 200 can communicate with an ultrasound system, which is configured to process data from a medium acquired by the probe. It can for example form part of an ultrasound system by ultrasonic waves. This system can, for example, be used in a medical context for the examination of organs and/or tissues.

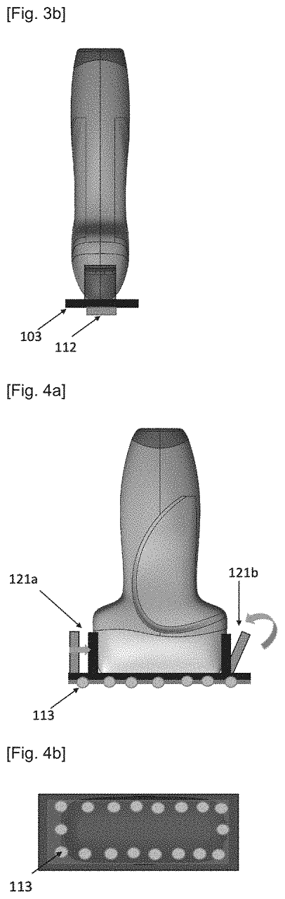

The probe can include a broader primary side (illustrated in FIG. 1, for example) and a narrower secondary side (illustrated in FIGS. 3a, 3b, for example).

The probe includes an envelope 205 which is a body containing the different components of the probe. The envelope 205 demarcates an interior from an exterior of the probe. The envelope is, for example, overall rigid to facilitate the manipulation of the probe. The envelope 205 includes, according to an example, a grip portion 206, overall ergonomic, by which the user manipulates the probe in a hand. The envelope 205 can also include a lower extremity 201, or, for instance, a cable or interface can link the probe to an external processing unit of the system.

In addition, the probe has a higher extremity or head part 202. Such higher extremity can be composed of a wave transmission surface 203 which is adapted to be in contact with the surface of a medium, such as the skin. The wave transmission surface 203 is shown in the Figures as being generally flat, or with a light curve, planar or in 2D. The wave transmission surface 203 can, however, present diverse forms in 3D, with possibly a pronounced curve.

The envelope 205 is made according to an example of one or several electrically insulating materials, such as plastic, for instance, of the ABS type. The envelope can consist of several assembled parts. For example, the envelope (without the wave transmission surface 203) could consist of a body of a single plastic item, leaving an opening adapted for receiving therein the emission and/or reception surface, thus connected and closing the envelope. The envelope 205 can be rigid or flexible, in whole or in part. According to an example, the emission and/or reception surface consists of one or several flexible polymer(s).

The probe 200 includes inside the envelope one or several transducers, for instance, several elements emitting and/or receiving acoustic waves set at a first extremity 202 of the probe 200. As per one option, it is possible that the probe 200 includes a single emitter and/or receptor element. According to one example, several emitter and/or receptor 120 elements comprise several piezoelectric elements. Multiple transducers or emitter and/or receptor elements can be placed so as to form a line or front of emission, or else an emission surface.

Moreover, the probe can include means of setting 204 on its narrower secondary sides which are configured to fasten any accessory or equipment.

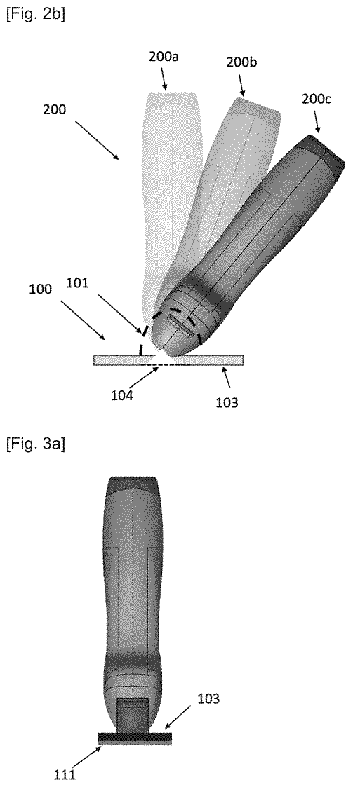

FIG. 2a schematically represents a perspective view of the first embodiment of the device 100 (in this example accompanying the probe) according to this disclosure. The device 100 can be configured to be linked to a probe 200. The device is also configured to be held against or to be in contact with the medium's surface during the examination of the medium. Thus, the device can be placed at the upper extremity 202 of the probe. More particularly, the device can be placed at the level of lens 203 of the probe, that is at the place where the ultrasound waves can be emitted by the probe.

The device is, in particular, configured to be linked to the probe 200 in a swiveling manner, so that an angle between the probe and the medium's surface can be adjusted by making the probe swivel in relation to the device. The device can remain in the same position of contact on the medium's surface during this swiveling movement.

The device can be configured to be able to swivel around at least one axis in relation to the probe, in particular, around an axis which corresponds to an axis of a line of transducers of the probe. Such axis can extend to the length of the first wider side of the probe, as illustrated, for example, in FIG. 2a. However, the device can also be configured to be able to swivel around two axes which, for example, extend along the wide primary side and in the direction of the waves, respectively.

The device 100 can be configured to be attached and/or fastened to the probe 200. For example, it can include two holding elements 101 which are configured to be attached to the probe in preserving the movement of rotation, particularly, to its means of fastening 204.

The device 100 can include a plate 103 configured to be placed against the surface of the medium and to stabilize the probe with a selected tilt angle between the probe and surface of the medium. In other words, the device 100 can have a flat surface which is placed on the surface of the medium to be examined (i.e., to be placed on the surface 203 of the probe). Such plate or surface 103 should be bigger than the transversal section of the surface 203 of the probe, so as to stabilize an inclined position of the probe in relation to the medium's surface. Furthermore, plate 103 can serve to supply homogeneous pressure on the medium's surface. Such pressure can thus lead to a homogeneous deformation of the medium. Said homogeneous deformation can be beneficial for different imaging modes, such as non-linear shear wave elastography (NL-SWE), as described above, and in WO2021116326A2.

The device 100 should, moreover, include an opening 104 with dimensions configured to enable the transmission of ultrasound waves emitted and/or received by the probe 200. Such opening 104 can, in particular, be arranged in plate 103, for example, in the form of a through hole. The dimensions of the opening can be predefined according to the size of the network of transducers inside the probe, in particular, according to the transversal section required for unobstructed transmission of waves.

The device 100, or at least plate 103, can consist of any rigid material, such as polymer or plastic. The plate material can be used, so that a homogeneous deformation of the medium can be obtained when the plate applies pressure on the medium's surface.

FIG. 2b schematically represents a first sectional view of the device in FIG. 2a, wherein a swiveling movement of the device in relation to the probe is illustrated. In a position by default 200a, the tilt angle can be 0°. This means that probe 200 can be placed perpendicularly to the surface of the device 100 and thus to a medium surface on which the device (that is plate 103) can be placed.

The device 100 is configured so that the probe can be swiveled in at least one or in both directions of swiveling, as illustrated in FIG. 2b, for instance, towards a position 200b and further towards a position 200c. The device 100 can thus be configured to allow a tilt angle up to 20°, or up to 30°, or even up to 45° (e.g., in each of the two directions of swiveling).

The means of setting 101 are only indicated schematically in FIG. 2b. Such means of setting can have different configurations as described in the context of other embodiments.

FIG. 2b also schematically demonstrates the opening 104 in the plate 103. Such opening 104 can be formed in a way that ensures a transmission of ultrasound waves in each possible tilt angle of the device. The opening can, for example, have the form of an inverted “V” (seen from the first sectional view as, for instance, schematically shown in FIG. 2b). Such form can thus present a narrower opening towards the probe and a broader opening towards the medium.

FIG. 3a schematically demonstrates a first lateral view of the second embodiment of the device according to this disclosure, wherein the device can have a coupling film. The second embodiment can correspond substantially to the first embodiment.

In addition, the device can include a means of coupling 111 on a surface facing the medium. Such part can be configured to support the transmission of ultrasound waves positioned in a zone where the ultrasound waves are emitted by the probe. Thanks to the means of coupling, it can be unnecessary to apply additional gel on the surface of the medium when the data is acquired. Due to its properties, the risk of the device sliding on the surface can be reduced. Likewise, the risk of infectious contamination due to the application of gel can also be reduced.

In the example of FIG. 3a, the means of coupling can consist of a gel pad and/or a film 111. This gel pad and/or this film 111 can cover the whole surface of plate 103 or only a part of the latter. For example, the gel pad can only cover or fill the opening 104 (not represented in FIG. 3a). Nonetheless, some gel can be applied—on the surface of the plate and/or in the opening (e.g., in the event where there is no film).

The means of coupling can also include a bi-material film 111 with an adherent part positioned in a peripheral area around the opening. The bi-material film 111 can also include a coupling part (e.g., a film) in a central area covering the opening.

FIG. 3b schematically demonstrates a first lateral view of the third embodiment of the device according to this disclosure, wherein the device can have gel. The third embodiment can correspond substantially to the second embodiment.

Also, the device can include a means of coupling 112 on a surface facing the medium. In the example provided in FIG. 3b, the means of coupling can consist of a film of gel 112 covering or filling the opening 104. The gel pad can, however, also cover the transversal section of the opening on the surface of the device facing the medium. It should be noted that FIG. 3b only illustrates this gel pad schematically.

The gel pad can notably be plane with the surface of plate 103 which faces the medium, that means that it does not protrude from this surface.

FIG. 4a schematically shows a second lateral view of the fourth embodiment of the device according to this disclosure, wherein the device can have a means of stabilization configured to optimize the stability of the device on the surface of the medium, for example, under the form of an adherent part with anti-sliding means to reduce the sliding of the device and adaptable points of attachment. FIG. 4b represents schematically a view from below of the device in FIG. 4a and, in particular, the disposition of the anti-slide points. Such view from below can specifically demonstrate the surface of the device which is configured to face the medium. The fourth embodiment can substantially correspond to any one of the above-mentioned embodiments.

The device can also include a means of stabilization configured to optimize the stability of the device on the surface of the medium, for example, under the form of adherent parts 113 on a surface of the device configured to be placed against the surface of the medium. In the example provided in FIGS. 4a and 4b, the adherent parts 113 can have the form of anti-slide points 113. The points 113 can, for instance, be constituted of a rubber material. As illustrated in FIG. 4b, the adherent parts can, in particular, be set in a peripheral area around the opening. In this way, the adherent parts can reliably prevent the sliding of the device on the medium's surface. At the same time, they do not disrupt the transmission of ultrasound waves through the opening.

The device can also include holding arms 121a, 121b configured to be attached to the device, for example, to its means of setting 204. The holding arms 121a, 121b can project from a rear surface of plate 103 which is opposed to the surface facing the medium.

The holding arms can, for instance, include stems projecting from the arms towards the holding means 204 of the probe. The stems can be formed so as to be able to be received and attached to the means of setting 204 in a swiveling manner.

The device can be configured for a rapid attachment and/or detachment of the probe. In the example illustrated, at least the holding arm 121a can be configured to be moved transversely on the rear surface in order to enable the attachment of the device to the probe bodies of different sizes. According to another example, the device can include a vice system. In this example, at least the holding arm 121b can be configured to be shifted in a swiveling manner on the rear surface of the plate, so that the holding arms can be clipped and unclipped on the probe. Subsequently, the device can be configured for an attachment and rapid release. However, the two holding arms can also have the shifting and/or swiveling function described above.

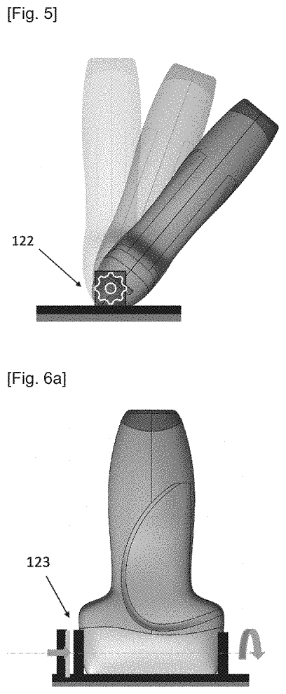

FIG. 5 schematically demonstrates a first lateral view of the fifth embodiment of the device according to this disclosure, wherein the device can have a mechanical means for swiveling through steps and/or indexation. The fifth embodiment can correspond substantially to any one of the above-mentioned embodiments.

As illustrated schematically, at least one of the holding arms can include a gear mechanism 122 (or any other mechanism) which is configured to allow solely a swiveling through steps/levels and/or indexing (i.e., through notches). In other words, the mechanism 122 can enable the attachment of the device to the probe uniquely at preestablished tilt angles.

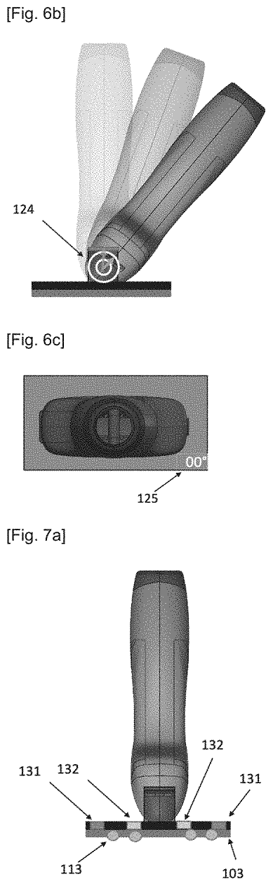

FIG. 6a shows schematically a second lateral view of the sixth embodiment of the device according to this disclosure, wherein the device can ensure a continuous swiveling. According to one option, the device can include an electromechanical means (or other mechanical means) configured for retaining in position and/or adjusting the angle of the device. The sixth embodiment can substantially correspond to one of the above-mentioned embodiments.

Moreover, at least one of the holding arms can include a clutch mechanism 123 (or any other mechanism) which is configured to ensure a continuous swiveling of the device in relation to the probe. In addition, the mechanism 123 can allow maintaining the device at any tilt angle selected, for example, by activating the clutch. Subsequently, the clutch mechanism can constitute an alternative to the gear mechanism 122 in FIG. 5.

The clutch mechanism can for example include a first clutch disc which is configured to be attached to the probe, so that a rotation of the first clutch disc in relation to the probe could be prevented. A second clutch disc can be attached to the plate 103, preventing any rotation of the second clutch disc in relation to the plate but allowing a transversal shift of the second clutch disc in relation to the plate. This transversal shift can ensure the activation of the clutch.

FIG. 6b schematically represents a first lateral view of the device in FIG. 6a with an optional tilt sensor 124. The tilt sensor can, for example, be integrated into the clutch mechanism 123. The tilt sensor can be configured to measure a tilt angle of the device in relation to the probe. The tilt sensor can be an electronic sensor.

FIG. 6c schematically represents a view from above of the device in FIGS. 6a and 6b with an optional tilt indicator 125. The tilt indicator can, for example, be an electronic display. The indicator can be configured to display the tilt angle, as measured by the tilt sensor 124.

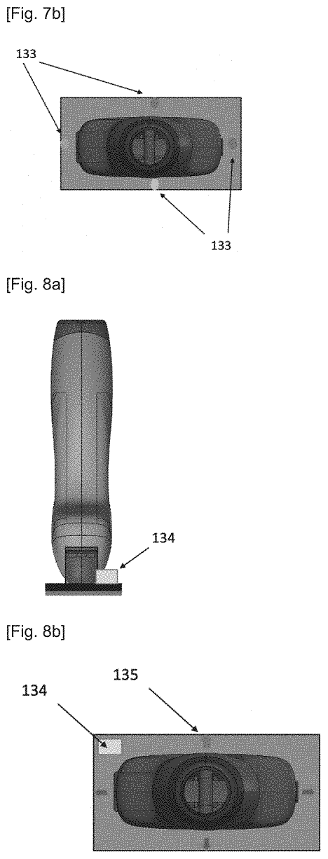

FIG. 7a schematically represents a first lateral view of the seventh embodiment of the device according to this disclosure, wherein the device can have pressure sensors 131 and/or temperature sensors 132. The seventh embodiment can substantially correspond to any one of the above-mentioned embodiments.

Therefore, the device can include at least one pressure sensor configured to measure the pressure that the device applies to the surface of the medium (in particular, its plate 102). Such pressure value can be used, for instance, as guide in the NL-SWE method (or, more generally, to determine a deformation level of the medium).

However, as demonstrated in FIG. 7a, the device can also include several pressure sensors 131. Such sensors 131 can be placed in different sections of the plate 103, for instance, around the opening 104. Thus, said sensors can be configured to measure local pressure applied by the respective section of the plate 103 to the medium's surface. In one example, the device can comprise four pressure sensors 131 (or any other number) configured to measure local pressure on the four sides of the plate 103 (e.g., of a rectangular form). Subsequently, the pressure sensors 131 can be situated so as to correspond to the positioning of the pressure indicators 133, as illustrated in FIG. 7b.

The device can also include one or several temperature sensors 132. Such sensors can be configured to measure the temperature of the medium's surface or of the probe's surface. The temperature measured can be used, for example, to monitor a possible heating of the medium due to the ultrasound waves emitted by the probe. For instance, in the event where the temperature exceeds a predefined value, a message or an alarm can be indicated to the user of the probe.

The device can also include adherent parts in the form of anti-slip points 113, as described in the context of FIGS. 4a and 4b.

FIG. 7b schematically represents a view from above of the device in FIG. 7a. FIG. 7b shows, in particular, the arrangement of the pressure indicators.

The device can include at least one pressure indicator 133 which is configured to indicate the pressure applied by the device, in particular, its plate 102, on the surface of the medium. Such pressure value can be determined, for example, by the pressure sensor 132. The pressure indicator can include or consist of a luminous element, for example, an LED. By changing its light color, the element of illumination can thus indicate a level of pressure.

The pressure indicator can be placed on the rear surface of the plate 103.

In another example, the light color can indicate a qualitative level of pressure. Such qualitative level of pressure can be determined, for example, by a comparison between a real pressure (e.g., measured by the pressure sensor) and a target pressure. Such target pressure can be determined in the context of the NL-SWE method, for example. In this method, different deformation levels of the medium can be required during the acquisition of data by the probe. On the basis of a required level of deformation, respective target pressure can be determined. In one example, the light colors can include: “green” (representing acceptable pressure), “red” (representing excessively high pressure), and any other color, such as yellow or blue (representing excessively low pressure).

However, as shown in FIG. 7b, the device can also include multiple pressure indicators 133. Such indicators 133 can be arranged in different areas of the plate 103, for example, around the area covered by the transversal section of the probe seen from above (cf. the view in FIG. 7b). Such indicators can be configured to indicate local pressure as measured by the pressure sensors 131 respectively arranged. In one example, the device can include four pressure indicators 133 arranged and configured to indicate local pressure on the four sides of the plate 103 (e.g., of a rectangular form).

FIG. 8a schematically represents a first lateral view of the eighth embodiment of the device according to this disclosure, wherein the device can include a positioning sensor and direction indicators. The eighth embodiment can substantially correspond to any one of the above-mentioned embodiments.

The device can include a positioning sensor 134, for example, in the form of an RFID label, magnetic field sensors, and/or one or several cameras. The positioning sensor can, for instance, transmit the data acquired to the system with which the probe communicates. The system can then implement a method of localization, as described below.

The RFID label can be configured to emit a signal through which the device can be localized. Such information on localization can be used jointly with external information from a target area on the surface of the support in order to determine a required direction of the device's shift. The target area can be a area where the data should be acquired by the probe. A target area can be identified by an algorithm implemented by computer and executed by the system, for example, an algorithm based on AI (artificial intelligence).

The camera can be configured to sweep the surface of the support. On the basis of the data from the camera, a current position of the device in relation to the surface of the medium can be determined. Such information on localization can be used jointly with external information from a target area on the medium's surface to determine a required direction of the device's shift.

It is also possible to use the data acquired by the probe for identifying the current position of the device in relation to the surface of the support. In this event, the positioning sensor 134 can be unnecessary.

FIG. 8b schematically represents a view from above of the device in FIG. 8a. In particular, FIG. 8b demonstrates direction indicators 135.

The device can include at least one direction indicator 135 which is configured to indicate a direction towards a target area. The direction indicator can include or consist of a display or a lighting element, for example, an LED. By changing its light color, the lighting element can indicate if a target area has been attained.

The direction indicator can be placed on the rear surface of the plate 103.

However, as illustrated in FIG. 8b, the device can also include multiple direction indicators 133. Such indicators 135 can be arranged in different areas of the plate 103, for example, around the area covered by the transversal section of the probe seen from above (cf. the view in FIG. 7b). Said indicators can be elements of lighting. In one example, the device can include four direction indicators 135 in the form of luminous arrows. The four direction indicators 135 can be located on the four sides of the plate 103 (e.g., in a rectangular form). By lighting the respective arrow(s), the user of the probe can be guided towards the target area.

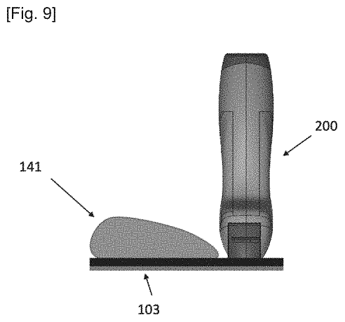

FIG. 9 schematically represents a first lateral view of the ninth embodiment of the device according to this disclosure, wherein the device can include an ergonomic support 141, such as a cushion and/or ergonomic handle. The ergonomic support can be configured in such a way that the user's hand can hold the probe and at the same time rest on the cushion. Subsequently, using the probe is more comfortable for the user, since the device is in a fixed position. At the same time, the user's hand can apply pressure via the ergonomic support on the surface of the support.

The ergonomic support 141 can be positioned on the rear surface of the plate 103. The plate can be made of any rigid material (e.g., plastic) and can then lead to homogeneous pressure, even if the ergonomic support is flexible.

Furthermore, the plate can be enlarged to provide a bigger surface of the rear surface on at least one side of the probe. Subsequently, the ergonomic support can be placed on such enlarged surface and can, therefore, also have a bigger and more comfortable size.

All these embodiments and other examples described above are merely non-exhaustive examples and can be combined and/or modified according to the scope of the following claims.

1. A device for an ultrasound probe for acquiring ultrasound data from a medium, wherein the device is configured to be linked to the probe in a swiveling manner, so that an angle between the probe and the surface of the medium can be at least one of adjusted and set by swiveling the device.

2. The device according to claim 1, also including:
at least one holding elements configured to attach the device to bodies of different probes.

3. The device according to claim 1, wherein the probe is at least one of configured to emit ultrasound waves and includes a transducer configured to emit ultrasounds waves on a primary side of the probe, and the device is configured to be at least one of positioned on and attached to the first side of the probe in a swiveling manner.

4. The device according to claim 1, further comprising:
an opening configured to enable the transmission of ultrasound waves.

5. The device according to claim 1, further comprising:
at least one of a plate and a flat surface configured to be placed against the surface of the medium and to stabilize the angle between the probe and the medium's surface.

6. The device according to claim 1, wherein the device comprises one of a gel pad and a film configured to enable the transmission of ultrasound waves.

7. The device according to claim 6, wherein the one of the gel pad and the film comprises an adhesive to attach at least one of the film the gel pad to the device.

8. The device according to claim 8, wherein the film includes a bi-material film with one or more adherent parts positioned in a peripheral area and a part configured for at least one of coupling the device to the medium and to enable the transmission of ultrasound waves and positioned in an area where ultrasound waves are emitted by the probe.

9. The device according to claim 1, wherein the device comprises at least one adherent part on a surface thereof, the at least one adherent part being configured to provide stability to the device on the surface of the medium.

10. The device according to claim 9, wherein the at least one adherent part is configured to reduce sliding of the device on the surface of the medium.

11. The device according to claim 1, wherein the device is configured to one of:
continuously adjust the angle; and
set the angle in a stepwise manner.

12. The device according to claim 1, also including at least one of:
a tilt sensor configured to measure the angle between the probe and the surface of the medium,
a display to indicate the angle,
a temperature sensor configured to measure the temperature of at least one of the surface of the medium and the surface of the probe,
a pressure sensor configured to measure the pressure applied to the surface of the medium by the device,
a pressure indicator configured to indicate a level of pressure applied to the surface of the medium, and
at least one of multiple local pressure sensors, each being configured to measure the pressure applied to the surface of the medium by a different area of the device, and multiple local pressure indicators, of which each pressure indicator is configured to indicate a level of local pressure.

13. The device according to claim 12, wherein at least one of the pressure indicator and local pressure indicators are placed on the opposite side of a surface of the device which is configured to be positioned against the medium.

14. The device according to claim 1, further comprising:
a position guiding system configured to indicate a target position of the probe on the surface of the medium, by guiding the probe or the user of the probe towards the target position.

15. The device according to claim 14, wherein the position guiding system includes at least one of:
direction indicators located on the surface of the device and configured to indicate a direction towards the target position, and
location indicators configured to localize the position of the device in relation to the medium.

16. The device according to claim 14, wherein the position guiding system is configured to communicate with an external processing unit to at least one of transmit localization data and receive data of at least one of a target position and a direction towards the target position.

17. The device according to claim 1, further comprising:
an ergonomic support configured such that a hand holding the probe can rest on the ergonomic support.

18. An ultrasound probe for acquiring ultrasound data from a medium, the ultrasound probe being configured to cooperate with a system intended for processing ultrasound data, the probe including a device according to claim 1.

19. An ultrasound system, including at least one of:
a device according to claim 1,
an ultrasound probe for acquiring data from a medium, and
location indicators configured to localize the current position of the device in relation to the medium.

20. A process for observing a medium through an imaging probe by using a device linked to the probe in a swiveling manner, including:
A. at least one of adjusting and setting the angle between the probe and the surface of the medium by swiveling the device; and
B. acquiring data from the medium by using the probe.

21. The process according to claim 20, further including at least one of:
C1. changing a level of deformation of the medium applied by the device, and
C2. changing the angle.

22. The process according to claim 20, including at least one of the following reiterations:
reiterating A and B;
reiterating B after at least one of C1 and C2;
reiterating B, and at least one of C1, C2; and
reiterating A and B, and at least one of C1, C2.

23. The device according to claim 11, wherein the device is configured to set the angle in the stepwise manner via a clutch.
